(19) United States
(12) Patent Application Publication
(10) Pub. No.: US 2016/0235881 A1
(43) Pub. Date: Aug. 18, 2016

(54) NON-ADHERENT HYDROGEL COATING FOR WOUND DRESSINGS AND METHODS FOR MAKING THE SAME

(71) Applicant: UNIVERSITY OF MANITOBA, Winnipeg (CA)

(72) Inventors: Sarvesh LOGSETTY, Manitoba (CA); Song LIU, Manitoba (CA)

(73) Assignee: UNIVERSITY OF MANITOBA, Manitoba (CA)

(21) Appl. No.: 15/026,191

(22) PCT Filed: Sep. 26, 2014
(86) PCT No.: PCT/CA2014/050927

§ 371 Date: Mar. 30, 2016

Related U.S. Application Data
(60) Provisional application No. 61/884,473, filed on Sep. 30, 2013.

(51) Int. Cl. A61L 15/60; B05D 3/06; A61L 15/32; A61L 15/26; A61L 15/44; A61L 15/18

(52) U.S. Cl. CPC A61L 15/60; A61L 15/44; A61L 15/18; A61L 15/32; A61L 15/26; B05D 3/065; A61L 2300/414; A61L 2300/104; A61L 2420/02; A61L 2300/404

(57) ABSTRACT

A method for preparing a hydrogel coating for a wound dressing to provide the wound dressing with non-adhesion properties without interfering with the bioactivity of the agents contained in the wound dressing. The hydrogel coating is formed by treating the substrate with O₂ plasma before applying a hydrogel precursor solution onto the substrate, and then curing the hydrogel precursor onto the substrate to form the hydrogel coating on the surface of the substrate. Hydrogel coatings for a wound dressing and wound dressings having a hydrogel coating are further described.

### FIELD OF THE INVENTION

The present disclosure relates to the field of wound dressings and, in particular, to a non-adherent hydrogel coating for wound dressings and methods for making the same.

### BACKGROUND OF THE INVENTION

Wound dressings are generally used to cover wounds in an effort to assist in the wound healing process. Wound dressings play a major role in wound care where dressings are essential to create optimal conditions for wound healing and improve patient comfort. In this respect, wound dressings function to protect the wound from microorganism infection, allow gas exchange, absorb exudate, and impart a moist environment to enhance epithelial regrowth. In effect, the environment created by the application of a wound dressing has been shown to improve epithelialization and wound healing, as well as effectively manage wound infection.

Newly formed tissue that has been regenerated during the healing process is particularly delicate and extremely sensitive to external influences. Such tissue is particularly vulnerable to damage resulting from removal and changing of the wound dressing. Moreover, adherence of the wound dressing to the wound bed can cause severe pain and discomfort to the patient as well as interfere with the healing process. In fact, such pain related to burn dressing adherence has been reported to result in severe depressive and posttraumatic stress symptoms (Browne A L, Andrews R, Schug S A et al (2011). Clinical Journal of Pain, 27(2), 136-45).

Wound adherence is a particular problem with many existing antimicrobial dressings. For example, dressings impregnated with silver compounds while being the mainstay of treatment for burn wounds, cause trauma upon removal due to adhesion to the wound bed. The commercially available silver based wound dressing known as Acticoat™, for example, requires the dressing to be wetted in order to reduce adhesion to the wound and to allow the dressing to be peeled off.

To avoid such pain related to burn dressing adherence, it is desirable that the dressing applied to the wound does not adhere to dried wound exudate, or in any coagulum formed, so as not to stick to the wound bed.

Hydrogel wound dressings have been used to provide moisture at a wound/dressing interface thereby avoiding adhesion of the dressing to the wound. Consequently, removal of a hydrogel dressing is usually neither painful nor detrimental to the healing process.

U.S. Pat. No. 4,438,258 relates to hydrogels which may be used as interfaces between damaged skin tissue and its external environment. As disclosed therein, hydrogels may be polymerized about some type of support, such as a mesh of nylon, used as an unsupported film, spun in fibers and woven into a fabric, or used as a powder.

United States Patent Publication No. 2013/0190672 describes electrospun zwitterionic monomers that are polymerized, electrospun, and crosslinked to form a non-woven fabric of nanofibers that can serve as a non-adherent and superabsorbent wound dressing.

United States Patent Publication No. 2013/0138068 describes a hydrogel laminate that can be used as a component of a wound dressing. In particular, a method is described for curing a hydrogel precursor within a fabric layer to produce a hydrogel laminate that is resistant to delamination.

Avoiding, or at least minimizing, pain related to burn dressing adherence continues to be challenging and a need remains for non-adherent wound dressings and methods for making existing wound dressings non-adherent.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

According to embodiments of the present disclosure, there is described herein a non-adherent hydrogel coating for wound dressings and methods for making the same. Specifically, the present disclosure relates to a method for growing, both chemically and physically, a hydrogel from a wound dressing substrate such that the hydrogel is grafted onto the surface of the substrate to form a part of the wound dressing. According to certain embodiments, the non-adherent hydrogel coating can be applied to a variety of wound dressing substrates, including without limitation, commercially available wound dressings.

In accordance with one aspect of the disclosure, there is described a method for forming a hydrogel coating on a wound dressing, comprising: (a) providing a substrate; (b) treating the substrate with O₂ plasma; (c) applying a hydrogel precursor solution onto the substrate; and (d) curing the hydrogel precursor onto the substrate to form the hydrogel coating on the surface of the substrate.

In accordance with another aspect of the disclosure, there is described a method for forming a hydrogel coating on a wound dressing, comprising: (a) providing a substrate, wherein the substrate is a wound dressing; (b) treating the substrate with O₂ plasma for between about 10 mins. to about 20 mins. to produce peroxide functional groups on the substrate surface; (c) loading a hydrogel precursor solution onto the plasma treated substrate, the precursor solution comprising acrylamide monomers, acrylic acid monomers, or a combination of acrylamide and acrylic acid monomers; (d) sandwiching the loaded substrate between two plates, wherein the two plates are positioned to define the thickness of the hydrogel therebetween; and (e) exposing the loaded substrate to ultraviolet irradiation for about 10 mins. to about 70 mins., wherein the ultraviolet irradiation is at an intensity of about 3 mw/cm² to about 120 mw/cm² UVA; wherein the hydrogel precursor solution is cured onto the substrate to form the hydrogel coating.

According to embodiments described herein, the substrate is a wound dressing and can further comprise an antimicrobial additive, a wound healing additive, or an antimicrobial and a wound healing additive.

According to further embodiments, the hydrogel precursor solution comprises acrylamide monomers and N,N′-methylene bisacrylamide as a cross-linking agent. According to other embodiments, the acrylamide monomers and the N,N′-methylene bisacrylamide are present in a weight ratio of from about 90:10 to about 99:1. According to further embodiments, the weight ratio of the acrylamide monomers and the N,N′-methylene bisacrylamide is about 98:2.

According to other embodiments, the method further comprises applying one or more antimicrobial agents, one or more wound healing agents, or one or more antimicrobial agents and one or more wound healing agents, to the hydrogel coating on the coated substrate, after the hydrogel coating is formed on the surface of the substrate. According to further embodiments, the method further comprises loading one or more antimicrobial agents, one or more wound healing agents, or one or more antimicrobial agents and one or more wound healing agents, onto the plasma treated substrate with the precursor solution, before curing the hydrogel precursor onto the substrate to form the hydrogel coating on the surface of the substrate.

In accordance with another aspect of the disclosure, there is described a hydrogel coating for a wound dressing formed by the methods described in the instant disclosure.

In accordance with a further aspect of the disclosure, there is described a wound dressing, comprising the hydrogel coating formed by the methods described herein.

In accordance with another aspect of the disclosure, there is described a wound dressing comprising a hydrogel coating on the surface of the wound dressing, the hydrogel coating comprising polymerized monomeric derivatives of acrylic acid, wherein the polymerized monomers are co-polymerized onto the surface of the wound dressing. According to embodiments described herein, the monomers can comprise acrylamide monomers, acrylic acid monomers, or a combination of acrylamide and acrylic acid monomers. According to further embodiments, the monomers comprise acrylamide monomers and N,N′-methylene bisacrylamide as a cross-linking agent. According to particular embodiments, the acrylamide monomers and the N,N′-methylene bisacrylamide are in a weight ratio of from about 90:10 to about 99:1. According to further embodiments, the weight ratio of the acrylamide monomers and the N,N′-methylene bisacrylamide is about 98:2.

In accordance with another aspect of the disclosure, there is described a hydrogel coating for a wound dressing comprising, polymerized monomeric derivatives of acrylic acid, wherein the polymerized monomers are co-polymerized onto the surface of the wound dressing. According to embodiments described herein, the monomers can comprise acrylamide monomers, acrylic acid monomers, or a combination of acrylamide and acrylic acid monomers. According to further embodiments, the monomers comprise acrylamide monomers and N,N′-methylene bisacrylamide as a cross-linking agent. According to particular embodiments, the acrylamide monomers and the N,N′-methylene bisacrylamide are in a weight ratio of from about 90:10 to about 99:1. According to further embodiments, the weight ratio of the acrylamide monomers and the N,N′-methylene bisacrylamide is about 98:2.

According to further embodiments of the present disclosure, the hydrogel coating can further comprise an antimicrobial additive, a wound healing additive, or an antimicrobial and a wound healing additive. According to certain embodiments, the wound healing additive is a growth factor. According to other embodiments, the antimicrobial additive is silver.

According to other embodiments, the hydrogel coating is about 20% to about 38% of the total weight of the wound dressing. According to further embodiments, the hydrogel coating has a swelling ratio of about 152% to about 365%.

According to certain embodiments, the hydrogel coating can reduce adherence of a wound dressing by up to about 95%. According to further embodiments, the hydrogel coating can reduce adherence of a wound dressing by up to about 60% to about 90%. According to other embodiments, the hydrogel coating can reduce adherence of a wound dressing by up to about 45% to about 65%.

### BRIEF DESCRIPTION OF THE DRAWINGS

These and other features of the invention will become more apparent in the following detailed description in which reference is made to the appended drawings.

FIG. 1 is a schematic illustrating the process of surface grafting hydrogel onto a PET dressing, according to embodiments of the present disclosure;

FIG. 2 is a graphical presentation of the peeling energy of hydrogel coated non-adhering dressings, according to embodiments of the present disclosure. Modified PET (1) was UV-irradiated for 50 min and modified PET (2) was UV-irradiated for 70 min;

FIG. 3 is a schematic illustrating the self-assembly of silver nanoparticles (AgNPs) loaded in a hydrogel according to embodiments of the present disclosure;

FIG. 4 is a digital image of AgNPs assembled on PAM-PET-3 (1st row) and untreated PET (2nd row), according to embodiments of the present disclosure;

FIG. 5 is a digital image of the AgNPs assembled on PAM-PET-3 shown in FIG. 4 at magnification of image: 9×, according to embodiments of the present disclosure;

FIGS. 6(a), (b), (c), and (d) are digital images illustrating the self-assembly capabilities of AgNPs on a) PET-PDMS, b) PET-PDMS-PAAc70-co-PAm30 Hydrogel, c) PET-PDMS-PAAc50-co-PAm50 Hydrogel and d) PET-PDMS-PAAc20-co-PAm80 Hydrogel fabrics, according to embodiments of the present disclosure;

FIG. 7 shows SEM images of the samples shown in FIG. 6, specifically: a) PET fabric and AgNPs loaded b) PET-PDMS, c) PET-PDMS-PAAc20-co-PAm80 Hydrogel, d) PET-PDMS-PAAc50-co-PAm50 Hydrogel and e) PET-PDMS-PAAc70-co-PAm30 Hydrogel, according to embodiments of the present disclosure;

FIG. 8 is a photograph of plates showing zone of inhibition results against P. aeruginosa and MRSA, where 1: PET; 2: PET-PAm; 3: PET-AgNPs; 4. PET-PAm-AgNPs; 5. Acticoat-Pam;

FIGS. 9, and 10 are photographs of plates showing the antibacterial effect of AgNPs in broth against P. aeruginosa and MRSA, respectively, over time;

FIGS. 11 and 12 are graphical presentations of the antibacterial activity of AgNPs-containing PET compared to modified PET against P. aeruginosa and MRSA, respectively;

FIG. 13 is a graphical presentation of the peeling energy (J/m²) of two commercial dressings (Acticoat™ and Silverlon™) before and after the deposition of polyacrylamide (PAM) hydrogel (PAM1: 9.8% (w/v) acrylamide (AM) and 0.2% (w/v) N,N′-methylene bisacrylamide (MBA), pressure applied onto the sandwiching glass plate: 0; PAM2: 9.8% (w/v) AM and 0.2% (w/v) MBA, pressure applied onto the sandwiching glass plate: 3254 Pa);

FIGS. 14A and 14B are graphical presentations of the antibacterial efficacy of the two commercial dressings (Acticoat™ and Silverlon™) before and after the deposition of polyacrylamide (PAM) hydrogel (PAM1: 9.8% (w/v) acrylamide (AM) and 0.2% (w/v) N,N′-methylene bisacrylamide (MBA), pressure applied onto the sandwiching glass plate: 0; PAM2: 9.8% (w/v) AM and 0.2% (w/v) MBA, pressure applied onto the sandwiching glass plate: 3254 Pa), FIG. 14A presents the zone of inhibition against MRSA (mm), and FIG. 14B presents the zone of inhibition against P. aeruginosa (mm);

FIG. 15 is a graphical presentation of the cumulative concentration of soluble Ag+ released from untreated Acticoat™ and Acticoat™ deposited with polyacrylamide (PAM) hydrogel (PAM1: 9.8% (w/v) acrylamide (AM) and 0.2% (w/v) N,N′-methylene bisacrylamide (MBA), pressure applied onto the sandwiching glass plate: 0; PAM2: 9.8% (w/v) AM and 0.2% (w/v) MBA, pressure applied onto the sandwiching glass plate: 3254 Pa); and

FIG. 16 is a graphical presentation of the relative cell viability (%) of fibroblasts exposed to both Acticoat™ and Acticoat™ deposited with polyacrylamide (PAM) hydrogel (PAM1: 9.8% (w/v) acrylamide (AM) and 0.2% (w/v) N,N′-methylene bisacrylamide (MBA), pressure applied onto the sandwiching glass plate: 0; PAM2: 9.8% (w/v) AM and 0.2% (w/v) MBA, pressure applied onto the sandwiching glass plate: 3254 Pa).

### DETAILED DESCRIPTION OF THE INVENTION

### Definitions

Unless defined otherwise, all technical and scientific terms used herein have the same meaning as commonly understood by one of ordinary skill in the art to which this invention belongs.

The terms “non-adherent”, “non-adhesion”, and “non-adhering” as used in the context of hydrogels according to embodiments of the present disclosure, refer to the ability of the hydrogel coated or grafted onto a substrate to render the substrate significantly non-adhering to wound tissue such that pain and/or destruction of the wound tissue is not caused by removal of the substrate from the wound.

As used herein, the term “about” refers to an approximately +/−10% variation from a given value. It is to be understood that such a variation is always included in any given value provided herein, whether or not it is specifically referred to.

The present disclosure describes exemplary embodiments relating to the preparation of hydrogel coatings useful in absorbent products such as wound dressings. Although the hydrogel coatings are being described in reference to wound dressings, it is readily understood that the hydrogels described herein can be used in other applications.

Hydrogels are three-dimensional networks of hydrophilic polymers, generally covalently or ionically cross-linked, which interact with aqueous solutions by swelling to some equilibrium value. These cross-linked gels are generally formed from synthetic polymers (such as polyvinylpyrrolidone, polyethyleneoxide, acrylate and methacrylate polymers and copolymers), multivalent alcohols (such as polyvinylalcohol), biopolymers (such as gelatin, agar), or combinations thereof.

Hydrogels, according to embodiments of the present disclosure, can be applied to a substrate to provide the substrate with non-adhesion properties. For the purpose of preparing wound dressings, as described in embodiments of the present disclosure, such substrates can include wound dressings that are commercially available. For example, wound dressings known in the art may include fabric, non-woven or woven polymeric films, metallic, paper, foam, and/or combinations thereof. Composite wound dressings are also available that include dressings containing bioactive agents. Typically, the bioactive agents are silver or other antimicrobial (e.g., antibacterial or antifungal) agents. Such agents are capable of destroying microbes, preventing the development of microbes or preventing the pathogenic action of microbes. According to embodiments of the present disclosure, bioactive agents may also include wound healing agents, for example, growth factors and anti-inflammatory agents. Coating such bioactive wound dressings with the hydrogel coating, according to embodiments of the present disclosure, provides the wound dressing with non-adhesion properties without interfering with the bioactivity of the agents contained in the wound dressing.

A preferred hydrogel for use according to embodiments of the present disclosure is cross-linked polyacrylamide (PAM). The hydrogel is grown both chemically and physically from a substrate that forms a part of a wound dressing. According to certain embodiments, the substrate is a wound dressing and the hydrogel is coated or grafted onto a surface of the wound dressing. In one embodiment, the substrate is a PET (polyethylene terephthalate) wound dressing. According to a further embodiment, the substrate is a wound dressing containing a bioactive agent, such as for example an antimicrobial, antifungal, or growth promoting agent. In certain embodiments, the bioactive agent may be silver or human epidermal growth factor. Examples of such bioactive dressings are known to those skilled in the art and include, for example, silver nanocrystal dressings such as Acticoat™ (Smith & Nephew) which has been treated with a silver deposit, silver impregnated dressings, and silver plated dressings such as Silverlon™ (Cura Surgical). In a preferred embodiment, the hydrogel is coated or grafted onto the surface of a bioactive dressing. According to further embodiments, the hydrogel is coated or grafted onto the surface of a silver dressing. According to other embodiments, the hydrogel is coated or grafted onto the surface of Acticoat™ wound dressing. According to further embodiments, the hydrogel is coated or grafted onto the surface of Silverlon™ wound dressing.

In further embodiments, the hydrogel is coated or grafted onto the surface of a wound dressing substrate prior to being treated with a bioactive agent. In this way, one or more bioactive agents can be loaded into the hydrogel grafted onto the surface of a wound dressing substrate to effectively impart a wound dressing substrate with both the non-adherence and bioactive functions derived from the loaded bioactive agent(s). In one embodiment, the bioactive agent is an antimicrobial, antifungal, or growth promoting agent. In certain embodiments, the bioactive agent may be silver or human epidermal growth factor. It is further contemplated that the one or more bioactive agent(s) may alternatively be combined with the hydrogel components and together applied to the wound dressing substrate. In alternative embodiments, the one or more bioactive agent(s) can be loaded onto the hydrogel coating that is already grafted onto a substrate.

As is known by those skilled in the art, the activity of certain bioactive agents may be altered or controlled by modulating its release through a variety of mechanisms. For example, the antibacterial activity of silver nanoparticles can be controlled by modulating Ag+ release through manipulation of oxygen availability, particle size, shape, and/or type of coating. Accordingly, it is contemplated that the rate and location of release of bioactive agents, such as Ag+ for example, may be defined and/or adjusted by combining with the characteristics of the hydrogel bound onto the wound dressing.

### Preparation of Hydrogel Coating

Grafting a hydrogel to a wound dressing, according to embodiments of the present disclosure, first requires activating the surface of the substrate by O₂ plasma treatment. Standard techniques of O₂ plasma treatment may be used to treat the substrate in preparation for application of the hydrogel coating.

By treating the substrate with O₂ plasma, the surface characteristics of the substrate material are altered. Specifically, O₂ plasma treatment produces peroxide functional groups on the surface of the substrate material. In one embodiment the substrate is treated with O₂ plasma for about 10 mins. to about 20 mins. In another embodiment, the substrate is treated with O₂ plasma for about 12 mins. to about 18 mins. In a further embodiment the substrate is treated with O₂ plasma for about 15 mins. to about 20 mins. In another embodiment, the substrate is treated with O₂ plasma for about 10 mins. In a further embodiment, the substrate is treated with O₂ plasma for about 20 mins.

Following treatment with O₂ plasma, a hydrogel precursor solution is applied and cured onto the substrate to form the hydrogel grafting. The hydrogel precursor solution, according to embodiments of the present disclosure, comprises a solution of monomeric compounds derivable from acrylic acid. Such monomeric compounds include, for example, acrylic acid (poly(acrylic acid) after polymerization), ethylene glycol methyl ether acrylate (poly(ethylene glycol) methyl ether acrylate after polymerization). In certain embodiments, the monomeric compounds have a Mn ranging from about 250 to about 5000 dalton.

According to embodiments, the hydrogel precursor solution comprises a monomer solution of acrylamide monomers, acrylic acid monomers, or a combination of acrylamide and acrylic acid monomers. According to a preferred embodiment, the hydrogel precursor solution comprises a monomer solution of acrylamide and N,N′-methylene bisacrylamide. The amount of monomer in the aqueous solution will be that required to produce a hydrogel of the desired content. In some embodiments, the monomer solution is provided at about 10% (w/v). In further embodiments, the monomer solution of acrylamide and N,N′-methylene bisacrylamide is provided in a weight ratio ranging from about 90:10 to about 99:1. In other embodiments, the weight ratio ranges from about 91:9 to about 98:2. In other embodiments, the weight ratio is from about 92:8 to about 97:3. In another embodiment the weight ratio is from about 95:5 to about 97:3. In a further embodiment, the weight ratio is 98:2.

The hydrogel precursor solution may be applied to the substrate using methods known in the art, however, according to some embodiments the hydrogel can be loaded onto the substrate and then sandwiched between two plates, for example, two pieces of glass. The distance allowed between the two plates determines the thickness of the hydrogel formed therebetween. According to such embodiments, the thickness of the hydrogel can be predetermined and controlled. In this way, application of the hydrogel coating is consistent and uniform throughout the substrate.

The hydrogel precursor solution is then polymerized onto the substrate by exposure to ultraviolet irradiation in a dose suitable to cross-link the monomers to form the hydrogel and to copolymerize those polymers and the plasma-treated substrate surface. The suitable dose will depend upon the nature of the monomers and the substrate used, and can be determined by one of skill in the art. Tests suggest that the UV irradiation dose should be at about 3 mw/cm² to 120 mw/cm² UVA for between about 10 to 70 mins. In one embodiment the dose is at about 3 mw/cm² UVA and applied for about 50 mins. In another embodiment the dose is at about 3 mw/cm² UVA and applied for about 70 mins. In a further embodiment the dose is at about 100 mw/cm² UVA and applied for about 10 mins. In another embodiment the dose is at about 100-120 mw/cm² UVA and applied for about 70 mins.

In a preferred embodiment, the hydrogel is grafted onto the substrate by a combination of treating the substrate with 20 mins. of O₂ plasma followed by 50-70 mins. of UVA treatment at a dose of 3 mw/cm² to cure the hydrogel precursor solution. In this way, the polymerized hydrogel is covalently bonded onto the substrate to form a uniform hydrogel coating.

### Loading of Bioactive Agents

As discussed above according to certain embodiments, the hydrogel described herein can facilitate the application of one or more bioactive agents onto the wound dressing substrate. For example, the one or more bioactive agent(s) can be loaded onto the substrate wound dressing before, after, or simultaneously with grafting the hydrogel onto the surface of the wound dressing substrate. In this way, a wound dressing substrate can be given both non-adherence and bioactive functions. In certain embodiments, the bioactive agent is an antimicrobial, such as for example silver nanoparticles. In other embodiments, the bioactive agent is a growth promoting agent that can assist in wound healing. Such bioactive agents are known to those skilled in the art. For example, growth promoting agents include, without limitation, human epidermal growth factor and anti-inflammatory agents.

In further embodiments of the disclosure, the hydrogel can impart time released activity of the embedded bioactive agent(s). As is known by those skilled in the art, the activity of certain bioactive agents may be altered or controlled by modulating its release through a variety of mechanisms including, as described herein, through the type of coating applied to the substrate wound dressing. Accordingly, it is contemplated that the rate and location of release of bioactive agents, may be controlled or defined by combining with the hydrogel bound onto the wound dressing.

### Hydrogel Coated Wound Dressings

Hydrogel coatings according to the present disclosure can be applied to a wound dressing in order to impart desired properties to the wound dressing. The amount of hydrogel coating applied to the surface of a wound dressing will vary depending on the type of hydrogel and the type of wound dressing. According to certain embodiments, a hydrogel coating can be applied to the surface of a wound dressing at a weight ratio of about 15% to about 40% of the total weight of the wound dressing. According to embodiments, a hydrogel coating can be applied at a weight ratio of about 25% to about 35% of the total weight of the wound dressing. According to further embodiments, a hydrogel coating can be applied at a weight ratio of about 20% to about 38% of the total weight of the wound dressing.

The properties imparted by the hydrogel coating are largely due to the ability of the hydrogel to interact with aqueous solutions. The hydrogel coating according to embodiments of the present disclosure exhibit a swelling ratio of up to about 365%. According to other embodiments, the hydrogel coating exhibits a swelling ratio of up to about 250%. According to further embodiments, the hydrogel coating exhibits a swelling ratio of up to about 150%. According to other embodiments, the hydrogel coating exhibits a swelling ratio of about 152% to about 365%.

The properties that can be imparted to a wound dressing include the ability of the hydrogel coating to reduce the adherency of the wound dressing. According to certain embodiments, the hydrogel coating can reduce wound dressing adherence by up to about 95%. According to other embodiments, the hydrogel coating can reduce adherence of the wound dressing by up to about 60% to about 90%. According to further embodiments, the hydrogel coating can reduce adherence of the wound dressing by up to about 45% to about 65%.

In addition to adherency, the hydrogel coating according to the present disclosure is non-inhibiting to antimicrobial or growth promoting additives. Accordingly, the hydrogel coating can be applied to wound dressings that contain bioactive agents without compromising the activity of such agents while imparting non-adherent properties to the wound dressing. According to embodiments, the hydrogel coating can be applied to wound dressings containing an antimicrobial, antifungal, and/or growth promoting agents.

In other embodiments, the hydrogel coating itself can be combined with bioactive agents to impart the bioactivity to the wound dressing. According to certain embodiments, one or more bioactive agents can be applied to the hydrogel coating on the surface of the wound dressing to impart the bioactivity. According to other embodiments, one or more bioactive agents can be combined with the hydrogel coating and together applied to the surface of the wound dressing.

The bioactive agents can vary but may include antimicrobial additives, such as silver, and/or wound healing additives, such as growth factors.

To gain a better understanding of the invention described herein, the following examples are set forth. It will be understood that these examples are intended to describe illustrative embodiments of the invention and are not intended to limit the scope of the invention in any way.

### EXAMPLES

### Example 1

### Preparation of Hydrogel Coating on PET Substrate

### Materials:

Acrylamide (AM) and N,N′-methylene bisacrylamide (MBA, crosslinker) were purchased from Sigma-Aldrich (Oakville, ON).

### Method:

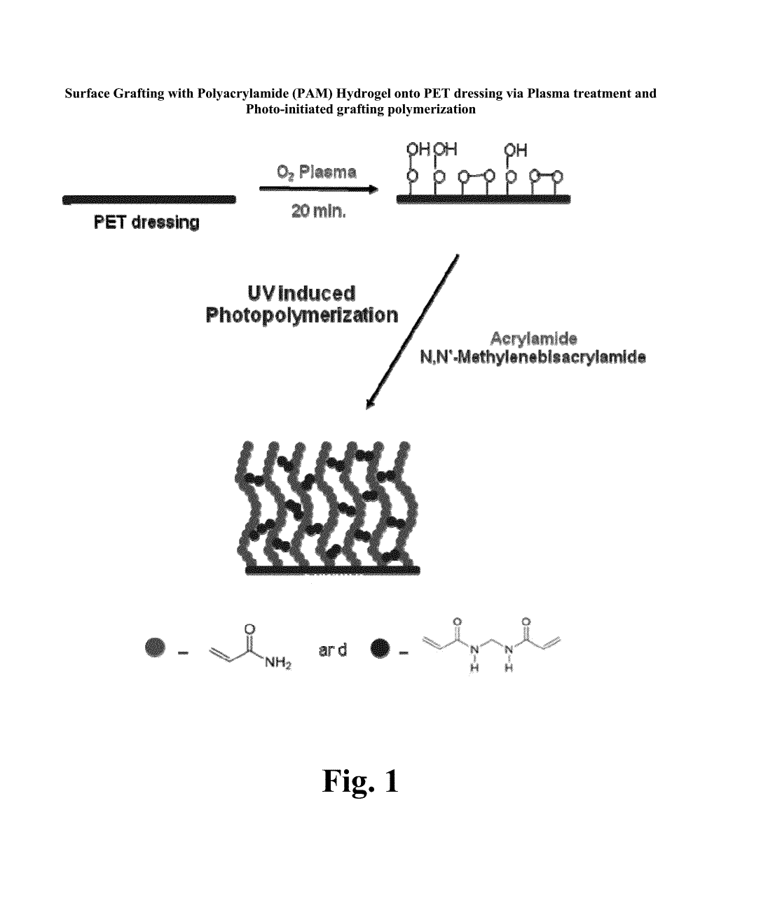

PET plain woven fabric (6×14 cm) was first extracted with deionized (DI) water for 24 hours to remove impurities before any treatment. The extracted PET fabric was treated with 13.56 Mhz O₂ plasma (300 W, flow rate of O₂: 2-4 standard cubic centimeter per minute) for 20 min to produce peroxide functional groups on the surface of the fabric. 3 mL monomer solution containing 9.8% (w/v) AM and 0.2% (w/v) MBA was uniformly placed onto the plasma treated PET fabric. Then the fabric was sandwiched by two glass plates prior to UV irradiation (365 nm, 3000 μw/cm²). Crosslinked polyacrylamide (PAM) hydrogel was grown from the surface of PET after UV irradiation (FIG. 1). The resultant PET fabric is referred to as “PET-PAM”.

### Example 2

### Preparation of Hydrogel Coating on Antimicrobial Wound Dressing

### Materials:

Acrylamide (AM) and N,N′-methylene bisacrylamide (MBA, crosslinker) were purchased from Sigma-Aldrich (Oakville, ON). Gelatin, silver nitrate (AgNO₃) and sodium citrate dehydrate (Na₃C₆H₅O₂.H₂O) were purchased from Fisher Scientific (Ottawa, ON). Sodium borohydride (NaBH₄) came from Acros (New Jersey, USA). Acticoat-3™ knit fabric was purchased from Smith & Nephew.

### Method:

A hydrogel coating was prepared for the commercially available antimicrobial dressing Acticoat-3™. The dressing was first treated with O₂ plasma for 20 min. A 10% (w/v) monomer solution was prepared with acrylamide and N,N′-methylene bisacrylamide at the weight ratio of 98:2. Then, the Acticoat™ dressing was loaded with 3 mL of the prepared monomer solution and sandwiched by two pieces of glass. The sandwich method can be used to control the thickness of hydrogel coating/grafting on the plasma treated PET fabric. 70 min UV irradiation (3 mW/cm² UVA) was used to initiate the co-polymerization of acrylamide and N,N′-methylene bisacrylamide to form the hydrogel on the surface of the dressing (FIG. 1).

The above method was carried out at different O₂ plasma treatment and UV irradiation treatment parameters. Initially, two UV intensities (3 and 100 mW/cm²) were used for the irradiation and gave good results in both cases. Further testing, however, indicated that the sample treated with 10 min O₂ plasma and 10 min UV irradiation (100 mw/cm²) was different than the sample treated with 20 min O₂ plasma and 50/70 min UV irradiation (3 mw/cm²). The hydrogel deposited on the Acticoat™ dressing was not uniform after 10 min UV irradiation with high intensity (100 mw/cm²), and some soft hydrogel remained on the sandwiching glass even after the setup was immersed in DI water before separating the two pieces of glass. Therefore, the lower intensity UV irradiation (3 mw/cm²) was used in the preparation of all hydrogel coated Acticoat™ dressings tested in the following experiments unless stated otherwise.

### Example 3

### Hydrogel Deposit and Water Absorption

### Hydrogel Deposit (Weight Gain):

The amount of hydrogel deposited was calculated by the weight increment of the PET fabrics after the polymerization as follows.

Weight increment=(Wₜ−W₀)/W₀×100%

where W₀ is the weight of untreated PET and Wₜ is the weight of sample after t min of UV irradiation.

### Water Absorption (Swelling Ratio):

Water absorption of the hydrogel coated dressings was assessed by comparing weights of the dry and hydrated samples. Samples were immersed in DI water for 5 min and centrifuged for 30 s at 2800 rpm to remove the excess water adsorbed on the surface. The swelling process was confirmed by change in weight: the ability for swelling is called “swelling ratio”.

Swelling ratio=(M₁−M₀)/M₀×100%

where M₀ is recorded as the weight of dry sample, and M₁ is the weight of swollen sample.

Measurement of swell ratio and weight changes after coating a PET dressing and an Acticoat™ dressing with hydrogel, showed that the hydrogel coating on Acticoat™ was slightly less in quantity compared to the untreated plain PET (see Table 1).

TABLE 1
Weight Gain and Swell Ratio of Wound Dressings

| | Weight Gain % | Swell Ratio % |
|---|---|---|
| PET | | 51.28 ± .84 |
| PET-HG | 38.46 ± 3.93 | 364.52 ± 21.69 |
| Acticoat ™ | | 41.40 ± 3.07 |
| Acticoat ™- | 20.51 ± 4.55 | 152.27 ± 9.01 |
| HG | | |

### Example 4

### Adhesion and Zone of Inhibition

### Peeling Force Test:

An in vitro model was chosen to mimic the environment between human skin and wound dressing: Briefly, A PTFE (polytetrafluoroethylene) window frame with an open area 16×60 mm² for gelatin casting was created. All the fabric samples (3×14 cm²) were soaked in DI water for 5 min, centrifuged for 30 s and then spread on a clean bench; one frame was placed onto each piece of fabric. 40 wt % gelatin solution was prepared in 70° C. DI water and then poured into the window frame. To simulate the wound desiccation process, the gelatin/fabric module was dried in the incubator at the skin temperature 32° C. for different time durations while maintaining a humid (75% RH) environment. Sulfuric acid (H₂SO₄) was used to maintain a constant humidity as per previously published protocols (Wilson R E. Humidity Control by Means of Sulfuric Acid solutions, with Critical Compilation of Vapor Pressure Data. The Journal of Industrial and Engineering Chemistry 1921; 13(4): 326-331). After a period of time (4, 8, 16, 24 h), the PTFE window was removed from the specimen and an Instron 5956 machine (Instron, Mass., USA) was used to peel the gelatin off the sample at a constant rate of 100 mm/min with 180° peeling angle. The five highest peaks were chosen to obtain the average force. Peeling energy per unit area (J/m²) was expressed as: θ=2P/b, where P was the average peeling force and b was the width of the gelatin strip (1.6 cm).

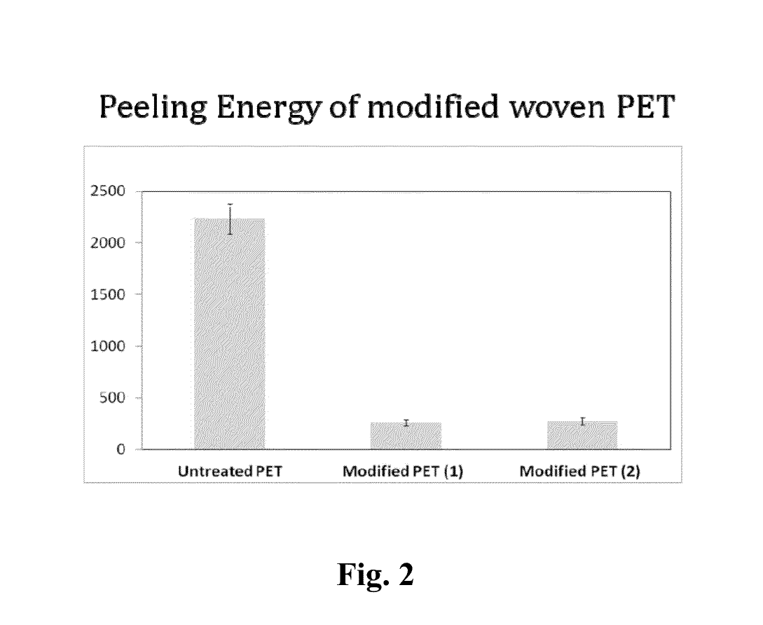

Initial work on adherence was done on an in vitro gelatin model with quantification of peel force by use of an Instron measuring device. The peel force of woven PET fabric was tested. As shown in FIG. 2, PET fabric (i.e., plain woven fabric #777 purchased from Testfabrics, Inc.) was coated with hydrogel. Modified woven #777 PET (1) was UV-irradiated for 50 min and modified woven #777 PET (2) was UV-irradiated for 70 min. The peeling energy data was collected after drying the fabric-gelatin module for 24 h at 32° C. in a humid (75% RH) environment. When modified with the hydrogel coating, the peel force of woven PET fabric was significantly reduced.

Prior to testing the adhesion of Acticoat™ dressing, raw PET knit fabric (prior to coating with silver to create Acticoat™) was tested and shown to have an adherence of 1721 J/m². By coating with hydrogel the adherence force was reduced to 113 J/m². Once this reduction in peel force was confirmed to be reproducible, the Acticoat™ dressing was tested. Standard Acticoat-3™ measured with a peel force of 2146 J/m²; coating with hydrogel reduced the peel force to 743 J/m² (a 65% reduction in force required) (see Table 2).

### Zone of Inhibition (ZoI):

The zone of inhibition study was carried out to test the antimicrobial activity of the hydrogel coated dressings. Standard operating procedures for testing ZoI were used. Two organisms were tested that are among the most common causes of burn wound infection and sepsis: Pseudomonas aeruginosa (Pa), and Methicillin Resistant Staphylococcus aureus (MRSA). As the hydrogel coating may have different properties if applied dry compared to when it is wetted, both applications were assessed. It was found that as expected there was no effective ZoI from the base PET material whether or not it was coated in hydrogel. There was also no significant difference between the original non-hydrogel coated Acticoat™, and the hydrogel coated Acticoat™ (dry or wet). The ZoI for Pa was 12.1 mm in coated and uncoated groups. For MRSA the ZoI was aprox 10.6 mm in the uncoated group compared to 10.4 mm in the coated group (see Table 2).

TABLE 2
Adhesion and ZoI Testing Results

| | Adherence | | |
|---|---|---|---|
| | (peeling force, | | Zone of Inhibition |
| | J/m²) | | mm |
| PET | 1721.5 ± 92.5 | | 0 |
| PET-HG | 113.8 ± 23.8 | | 0 |
| Acticoat ™ | 2146.3 ± 448.8 | DRY | Pa 12.13 ± .48 |
| | | | MRSA 10.38 ± .25 |
| | | WET | Pa 12.90 ± .42 |
| | | | MRSA10.65 ± .51 |
| Acticoat ™- | 743.8 ± 67.5 | DRY | Pa 12.13 ± .47 |
| HG | | | |
| | | | MRSA 10.23 ± .53 |
| | | WET | Pa 13.05 ± .33 |
| | | | MRSA 10.45 ± .67 |

### Example 5

### Loading of Bioactive Agent onto Hydrogel-Coated Wound Dressing

A silicone coated PET fabric was used as the substrate and the hydrogel covalently grafted onto the surface by plasma treatment surface activation and photopolymerization (“PET-PDMS-PAAc-co-PAm Hyd”). The ability of AgNPs to self-assemble on the treated substrate and maintain bioactivity was then assessed.

### Method:

8.5 mg AgNO₃ was dissolved in 45 ml deionized water, then 14.7 mg Na₃C₆H₅O₇.2H₂O was added under continuous stirring, and finally 5 ml NaBH₄ solution (9.5 mg) was rapidly added under vigorous stirring. After 1 h, the pH of the AgNPs solution was adjusted to 5.

PET-PDMS-PAAc-PAm Hyd (2×2 cm) was immersed into the AgNPs solution and shaken. After 3 h, the fabric was washed with deionized water several times and dried at room temperature.

### Self Assembly of AgNPs on PET-PDMS-PAAc-co-PAm Hydrogel Fabrics:

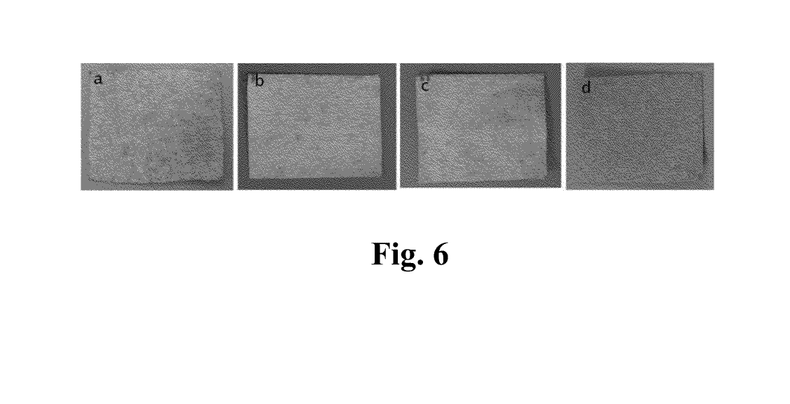

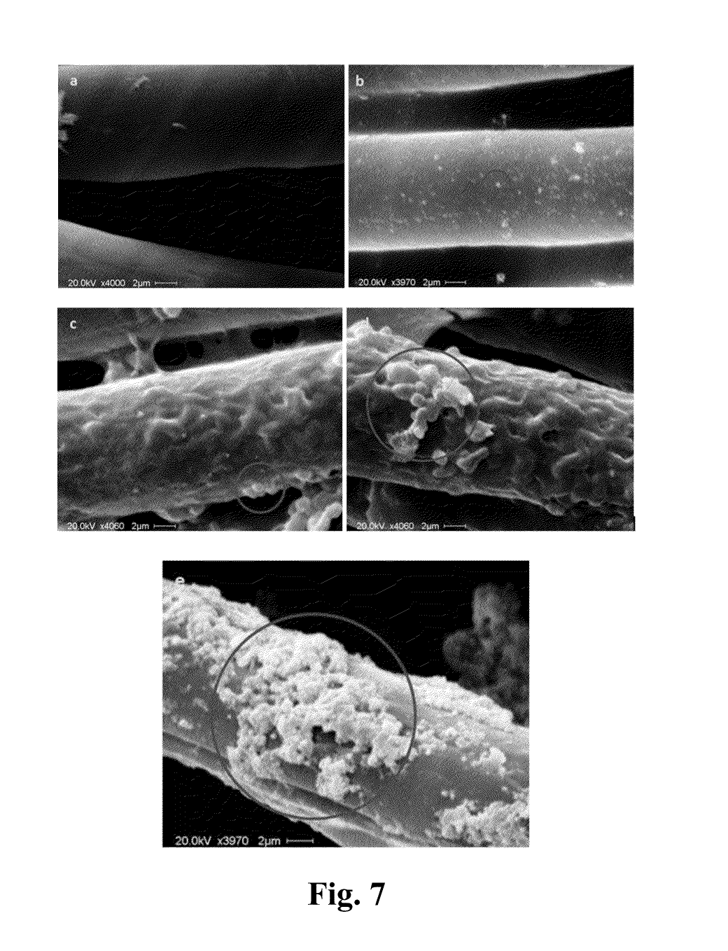

In order to confirm the self-assembly of AgNPs on the hydrogel, the blank experiment was carried out without hydrogel on the PET-PDMS (Silicone coated PET fabric). As shown in FIG. 6a change in colour (gray) of PET-PDMS fabric was observed and this preliminary result concludes that the small amount of AgNPs was adsorbed on the surface of PDMS. Referring to FIG. 6d, shows that AgNPs were successfully loaded on a hydrogel coated substrate comprising 80% PAm (polyacrylamide monomers) and 20% PAAc (polyacrylic acid monomers) hyd on PET-PDMS fabric. The AgNPs were uniformly loaded, confirming that the hydrogel formation was also uniformly grafted onto the substrate surface. FIG. 6b, however, shows that the AgNPs were retarded by fabrics with a higher amount of PAAc (70%). This is due to the repulsion of the acid groups on the fabric and on AgNPs. Furthermore, FIG. 6c shows that the AgNPs were not loaded even at 50% PAAc. SEM imaging further confirmed the above observations (FIG. 7).

### Example 6

### Loading of Bioactive Agent onto Hydrogel-Coated Wound Dressing

The loading capacity of a hydrogel-coated wound dressing was assessed using silver nanoparticles (“AgNPs”) as an exemplary bioactive agent. A PET fabric having a hydrogel covalently grafted onto the surface (“PAM-PET”) was used in this study. The ability of AgNPs to self-assemble on the treated substrate was then assessed.

### Method:

8.5 mg AgNO₃ was dissolved in 45 ml deionized water, then 14.7 mg Na₃C₆H₅O₇.2H₂O was added under continuous stirring, and finally 5 ml NaBH₄ solution (9.5 mg) was rapidly added under vigorous stirring. After 1 h, the pH of the AgNPs solution was adjusted to 5.

PAM-PET (2×2 cm) was immersed into the AgNPs solution and shaken. After 3 h, the fabric was washed with deionized water several times and dried at room temperature.

Three samples were tested for this study. The PAM-PET-Ag(1, 2, 3) designation is based on the original PET-PAM characteristics onto which the AgNPs were loaded. See Table 3 below.

TABLE 3
Concentrations of Monomer, Crosslinker and Initiator in the Surface
Modification of PET

| | Concentration (mol/L) | | |
|---|---|---|---|
| | Monomer | Crosslinker | Initiator |
| Samples | AM | MBA | BP |
| PAM-PET-1 | 3 | 0.45 | 0.055 |
| PAM-PET-2 | 3.6 | 0.45 | 0.055 |
| PAM-PET-3 | 4.5 | 0.45 | 0.055 |

### Self Assembly of AgNPs on PAM-PET Hydrogel Fabrics:

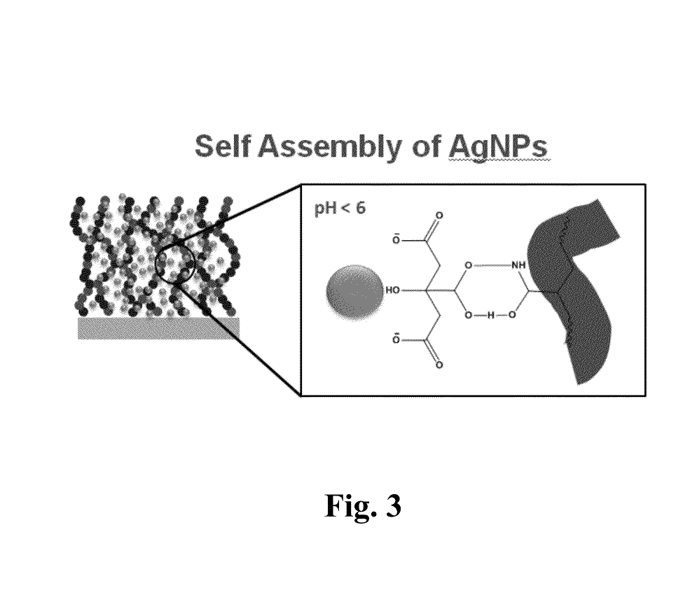

The AgNPs self-assembled on the PAM-PET fabric by hydrogen bonding which was achieved between carboxylic groups in sodium citrate and amide moieties (FIG. 3). Through interfacial dimeric hydrogen bonding, AgNPs were successfully assembled.

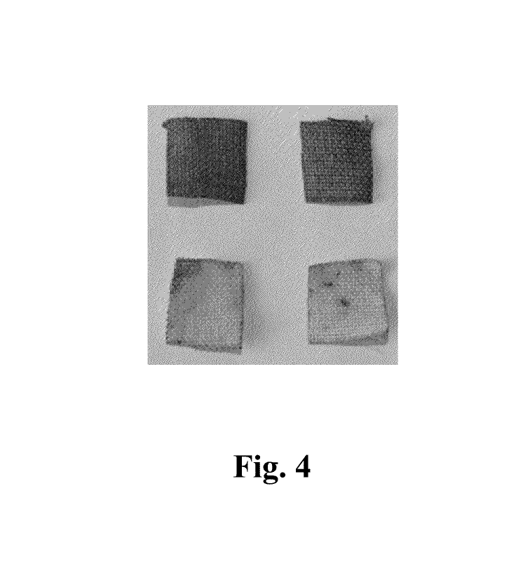

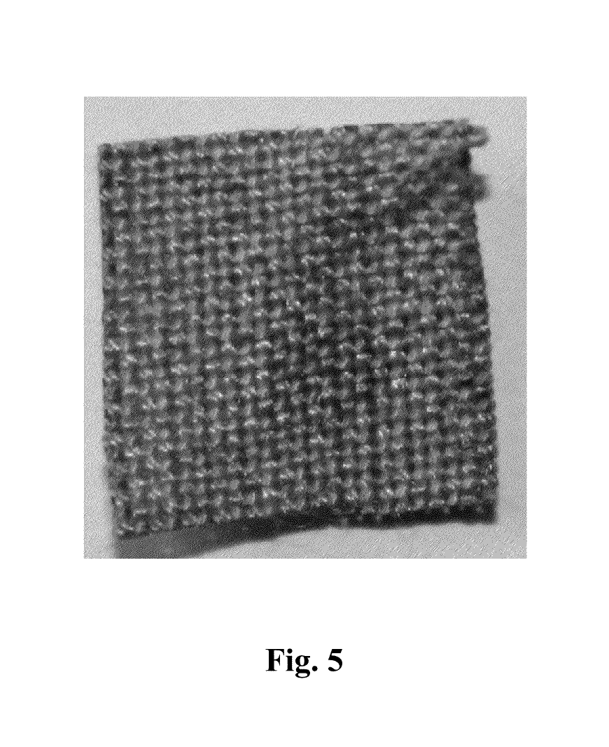

As shown in FIGS. 4 and 5, the fabric was dark brown with particles on it while the untreated samples remained white with only limited quantities adsorbed between fibers (FIG. 4). Because of numerous AgNPs, the PAM-PET-Ag-3 was shiny as metal (FIG. 5).

### Determination of AgNPs Content:

The amount of AgNPs on the hydrogel-coated fabrics was determined using a thermogravimetric analyzer (TGA, TA Instruments model XYZ). Around 3 mg of AgNPs loaded onto a hydrogel-coated fabric was placed into a TGA platinum pan. The temperature is ramped at 5° C./min to 800° C. under oxygen environment.

The mass percentage of loaded silver was analyzed by TGA. PET served as a control sample and the silver percentages of PAM-PET-Ag-3, as an example, was the mass change difference between control sample and modified sample. The mass change of untreated PET was 98.39% and that of PAM-PET-Ag-3 was 94.61% and therefore, the silver content in PAM-PET-Ag-3 was 3.78%, which was higher than PAM-PET-Ag-1 (3.02%) and PAM-PET-Ag-2 (2.90%).

### Example 7

### Bioactivity of Bioactive Agent-Loaded-Hydrogel

### Bacterial Zone of Inhibition:

Two bacterial species, Gram negative P. aeruginosa with a GFP expressing plasmid and Gram positive S. epidermidis, were used in a zone of inhibition study. The AgNPs loaded PET-PAM fabrics and control samples, all with diameter of 9 mm, were soaked in water for 24 h, sterilized with UV, washed with 70% ethanol three times, and rinsed with sterile water three times for these bacterial studies.

### Zone of Inhibition:

The zone of inhibition study was carried out to test the antimicrobial activity of the AgNPs loaded PET-Pam. The bacterial lawns were prepared by inoculation of agar plates with P. aeruginosa and incubation at 37° C. overnight. PET, PET-Pam and PET loaded with AgNPs (PET-AgNPs) were used as controls. Using a modified Kirby Bauer technique, the samples and the controls were placed on the bacterial lawns and incubated at 37° C. for 24 h. The zone of inhibition was then measured around the above fabrics.

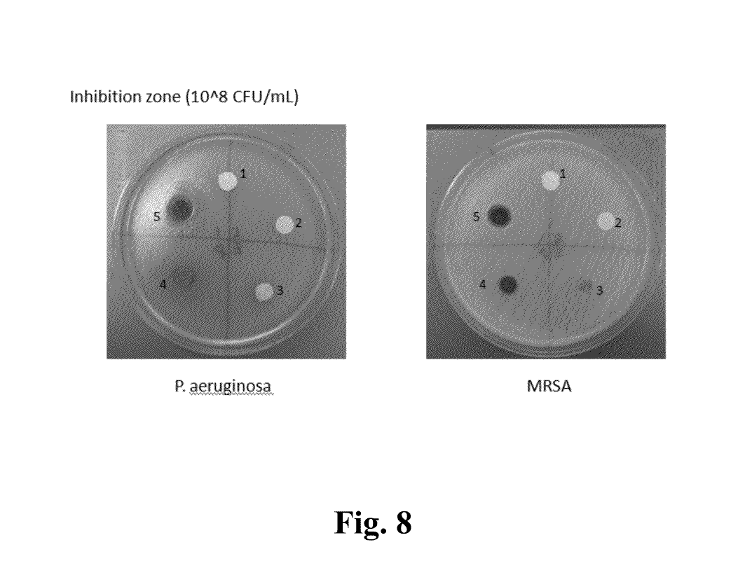

The results are shown in FIG. 8, where Sample 1: PET; 2: PET-PAm; 3: PET-AgNPs; 4. PET-PAm-AgNPs; 5. Acticoat-Pam. The bioactivity of the samples against P. aeruginosa is reflected in the respective zone of inhibition. The diameter of the inhibition zone of Acticoat-PAm (sample 5) was 11-11.5 cm, and the diameter of the inhibition zone of PET-PAm-AgNPs (sample 4) was 8.5-9 cm.

For MRSA, the diameter of the inhibition zone of Acticoat-PAm (sample 5) was 9.5-10 cm, and no inhibition zone was observed for other samples.

### Antibacterial Effect of AgNPs:

Briefly, a 20 mL bacterial suspension (10̂6 CFU/mL) was prepared and fabric samples prepared with the size of 1*1 cm². Timing started immediately after adding the fabric samples into the bacterial solution and shaken on the Titer plate shaker at 120 rpm. After different time durations, 1 mL bacteria solution was taken into 1 mL PBS (dilution here was supposed to quench the AgNPs) and then diluted 10 times (100 uL bacterial solution was taken into 900 uL PBS) each in due succession before plating on the agar plates.

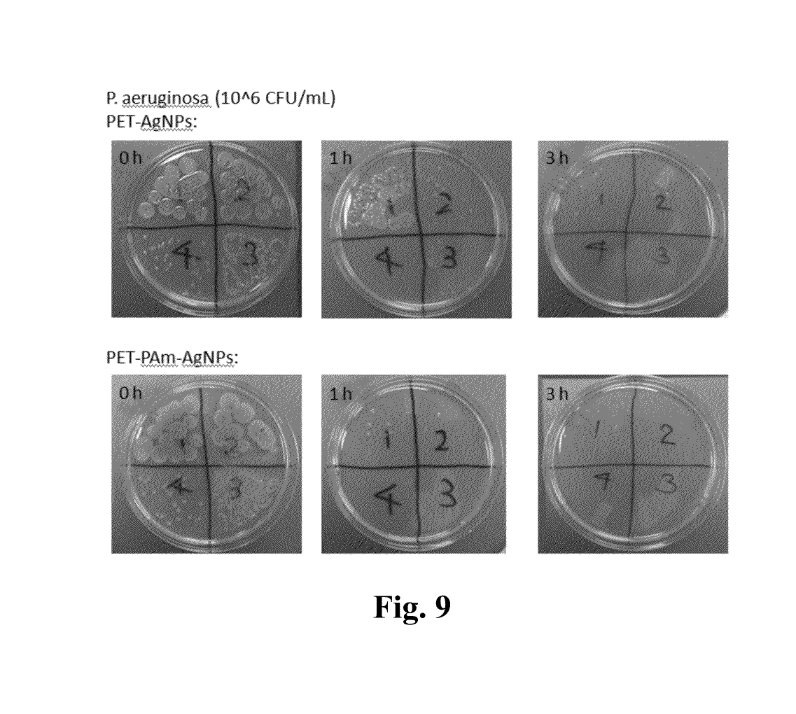

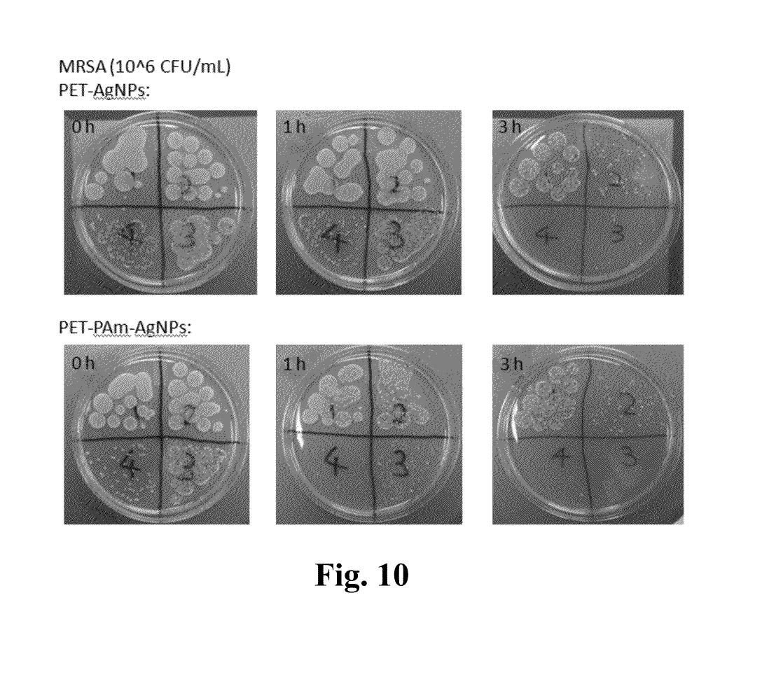

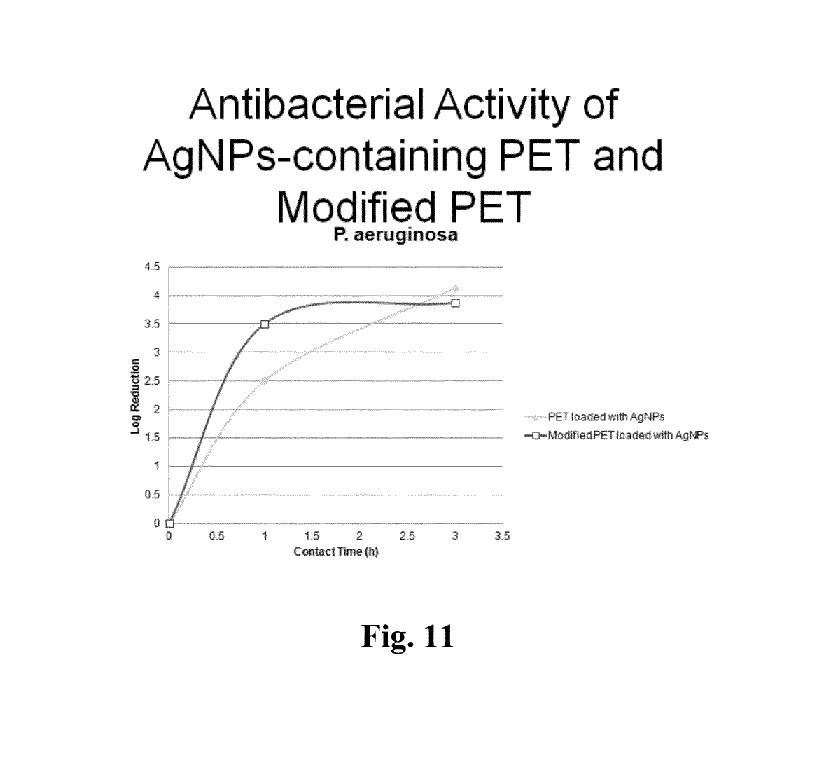

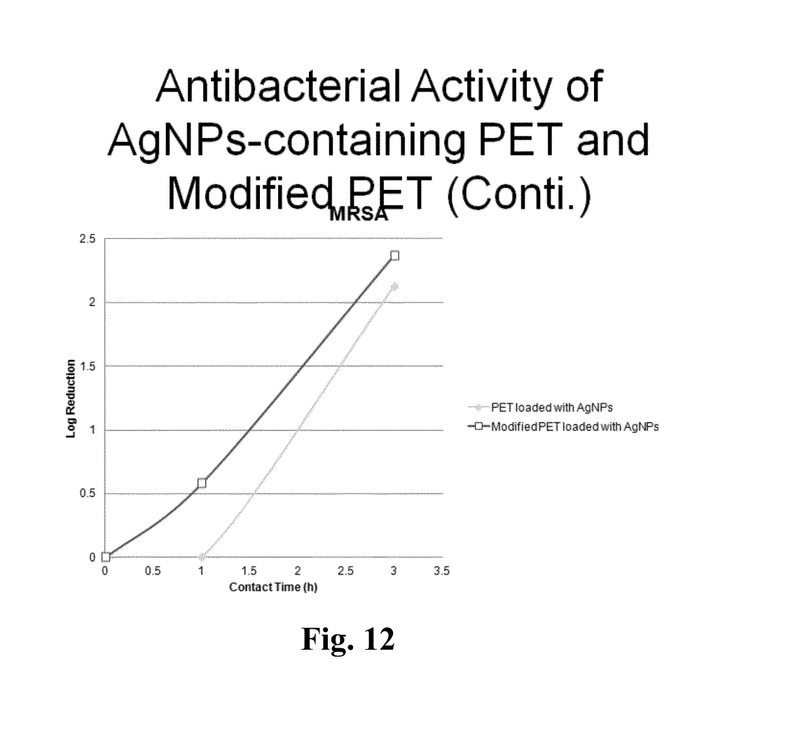

The results are shown in FIGS. 9 and 10. As reflected in FIGS. 9 and 10, it is demonstrated that the silver did kill the bacteria over time. These results are further graphically reflected as log kill ratios in FIGS. 11 and 12.

### Example 8

### Commercially Available Antibacterial Wound Dressings

The applicability of hydrogel coatings, described herein, for improving the adherent profile of commercially available antibacterial wound dressings was further explored. In particular, the effect of depositing a layer of poly (acrylamide) (PAM) hydrogel onto a commercially available antibacterial dressing was observed.

A layer of PAM hydrogel was grafted onto two commercial AgNP antibacterial dressings (Acticoat™ Flex 3 and Silverlon™) using plasma-induced graft polymerization described as follows.

Pieces of 6×14 cm (14 would be the less stretchable direction of fabric) of Acticoat™ Flex 3 (purchased from Smith & Nephew) and Silverlon™ (purchased from Argentum Medical, LLC, Geneva, Ill.) wound dressings were treated with O₂ plasma (Flecto10-PC-MFC, PLASMA technology, Germany) at a flow rate of 24-26 (sccm) for 20 minutes. Subsequently fabrics were treated with 5 mL monomer solution dropwise. The monomer solution was a mixture of 1.38 (mol/L) Acrylamide (AM) and 0.013 (mol/L) N,N′-methylene bisacrylamide (BAM) (purchased from Sigma-Aldrich (Oakville, ON)) in distilled water (the solution was deoxygenated with nitrogen before usage). The fabrics were then sandwiched between two glass plates and exposed to UV irradiation (Irradiation intensity: 100 mW/cm2) (Intelli-Ram 400 Shuttered UV flood light UV338) for 15 minutes. Samples were then rinsed with distilled water and the un-grafted monomer was removed by shaking the samples for two hours in reciprocal shaking bath (Precision 2870, Thermo Scientific Fisher) at 65° C. and 150 RPM. The samples were then dried in oven at 105° C. for 30 minutes and stored in desiccator for 24 hours. (referred to as PAM1).

In order to test the effect of hydrogel thickness on the properties of wound dressing, a similar treatment was done on another batch of samples while placing 1626.8 (pa) pressure on both sides of the upper glass slide during the UV exposure procedure. (referred to as PAM2).

### Adhesion:

The adherency profile of the coated dressings was tested using the peeling force model described above (Example 4).

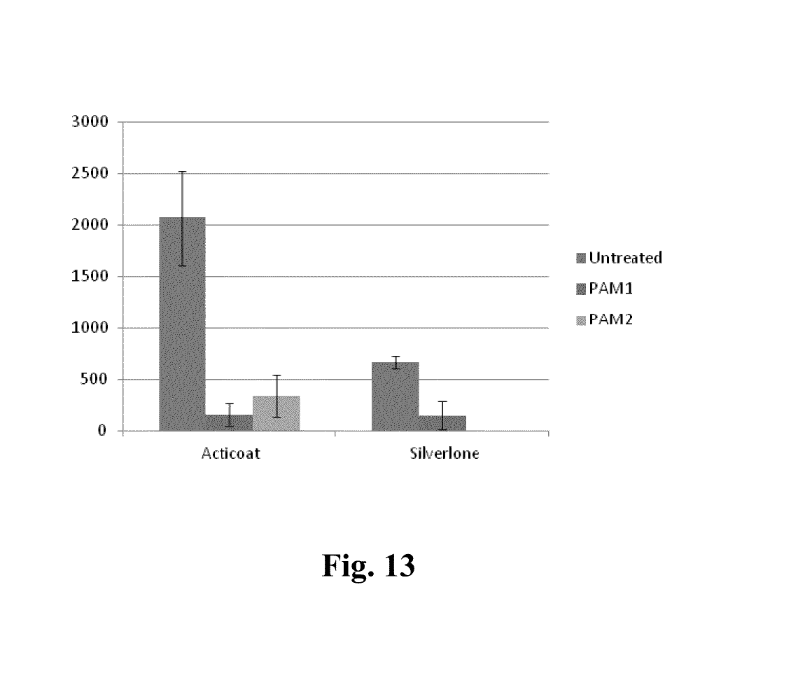

As shown in FIG. 13, both untreated Acticoat™ and Silverlon™ showed high peeling energy (2070±453 J/m² and 669±68 J/m², respectively). Deposition of PAM hydrogel reduced the peeling energy to 158±119 J/m² for Acticoat™ and to 155±138 J/m² for Silverlon™.

### Antibacterial Efficacy:

To study the impact of hydrogel deposition on the antibacterial efficacy of the two commercial dressings, the agar diffusion test was conducted in accordance with the method described above (Example 4). In general, both commercial dressings were challenged with two clinically retrieved bacterial strains (MRSA and multi-drug resistant (MDR) P. aeruginosa) in an antimicrobial disk susceptibility test.

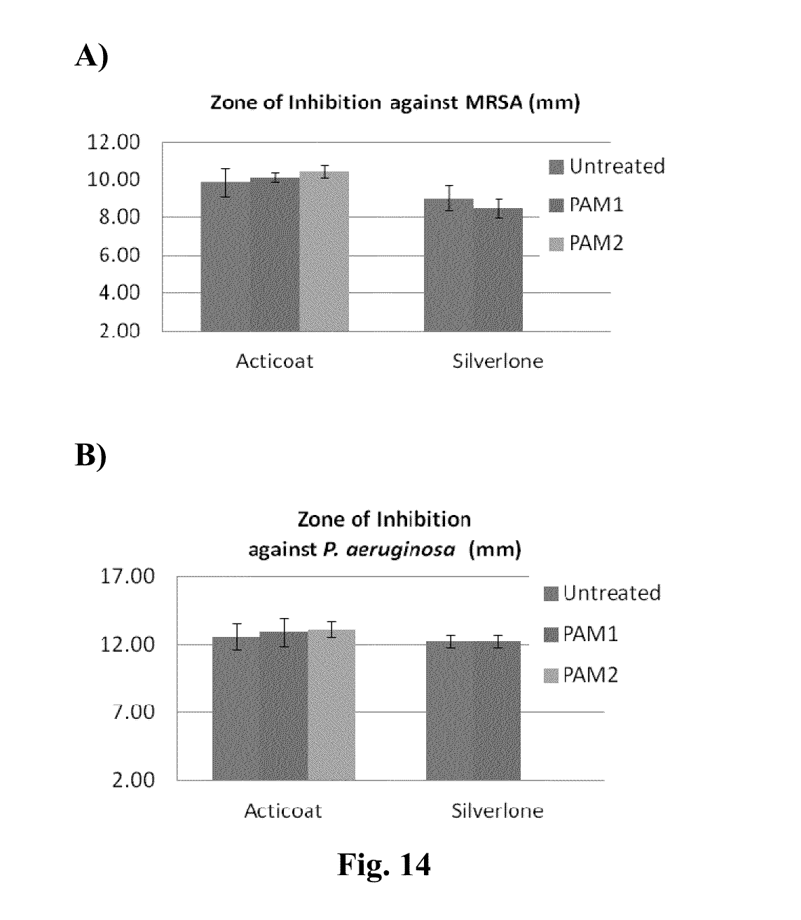

The results of the agar diffusion test (FIG. 14) show that the deposition of hydrogel caused a marginal increase of the inhibition zone (p>0.05) in Acticoat™ for both bacteria. No significant change was observed between the Silverlon™ with PAM hydrogel deposition, and without.

### Silver Ion Release:

To shine some light on the impact of hydrogel grafting on the release of silver ion (Ag+), inductively coupled plasma atomic emission spectroscopy (ICP-OES) was used to quantify the amount of Ag+ released.

To monitor the release of silver ions, (2.5×2.5 cm²) rectangular pieces (0.075 gram) from the Acticoat Flex 3 dressings (untreated and treated) were transferred into sterile vials. Ultra-pure water was used as media for silver release in a 1:100 w/W ratio. The vials were capped and placed in incubator at 32° C. from 30 minutes, 2 hours to the maximum application time of dressing which was 2-3 days (each condition was done in triplicate). After proper timings solutions were gently mixed by pipetting up and down and one mL aliquots was sampled from each vial and kept in Eppendorf tubes. After each time sampling the vials were topped up to keep the same volume. Immediately after sampling the Eppendorf tubes were stored at −20° C. Prior to the ICP-OES, samples were centrifuged for 10 min at 14,000 rpm for the removal of insoluble Ag precipitates. Samples were submitted for the ICP-OES test and the test was done using Varian (Agilent) 725ES ICP-OES instrument. The test was done after diluting each of the 1 mL aliquots into 4 mL (10%) HNO₃.

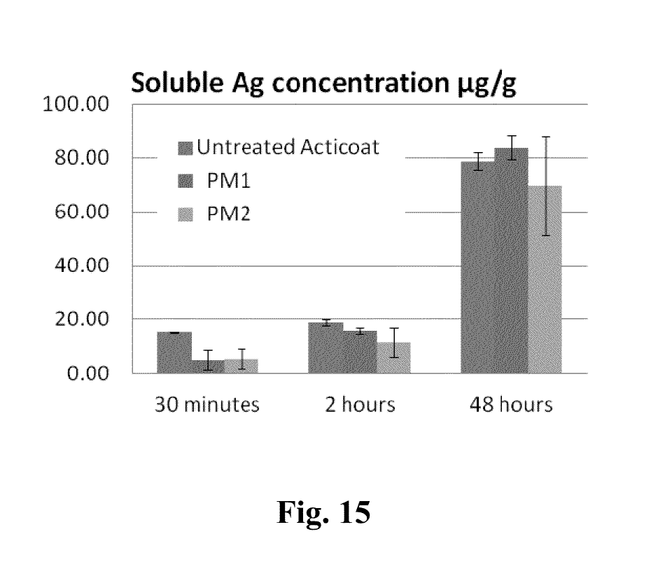

FIG. 15 presents the cumulative concentration of soluble Ag+ released from the Acticoat™ dressings after 30 min, 2 h and 48 h of incubation. The amount of released Ag+ from hydrogel deposited Acticoat™ at 30 min is significantly less than that from untreated Acticoat™ (P<0.05). The difference, however, becomes smaller over time. Hydrogel deposited Acticoat™ (PAM1) even shows a directional increase of cumulative concentration of soluble Ag+ as compared with untreated Acticoat™. This observation correlates with the similar antibacterial efficacy observed in the agar diffusion results for Acticoat™ before and after hydrogel coating, where the dressing samples were allowed to contact the bacterial lawn for 16-18 hours in the agar diffusion test.

### Cytotoxicity:

The cytotoxicity of samples to human neonatal fibroblast cells was further evaluated by a standard MTT assay.

In vitro cytotoxicity testing involved growing ACTT-PCS-201 neonatal human dermal fibroblasts in fibroblast basal medium supplemented with fibroblast growth kit—low serum (ACTT-PCS-201-041) and incubated in 5 vol % CO₂ under humidified conditions at 37° C. After reaching 80% confluency, the cells were trypsinized, quantified with a haemocytometer, seeded onto tissue culture-treated polystyrene 24-well plates at a final density of 10⁵ cells/mL, and incubated at 37° C. for 24 h. After 3 days, after reaching 70-80% confluence the experimental treatment was applied. The media was aspirated and 1.1 mL of culture medium added to each of the culture wells. Dressings were cut into round shape disks (0.7 cm diameter) sizes and sterilized by autoclaving. The dressings were then presoaked in 0.4 mL of saline/DI water for 10 minutes at 37° C. in the incubator. In the next step the dressings (0.7 cm diameter) together with 0.4 mL saline/water was added to each well. Addition of 1.5 mL of the plain solute (0.4 mL (saline/DI water)+1.1 mL culture medium) without dressing is regarded as a positive control. The cells were then incubated at 37° C. in a humidified atmosphere of 5% CO₂ for 24 hours. After 24 hours, the dressings were removed and by using (MTT) assay the cell viability determined. Three 100 μL aliquots of each well were then transferred to a 96-well plate. Cell viability was evaluated by spectrophotometry at 570 nm wavelength.

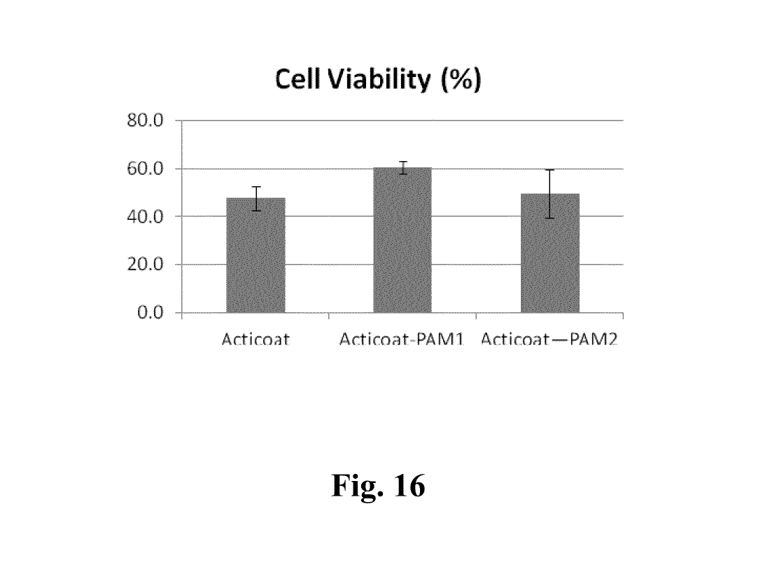

The cytotoxicity of the Acticoat™ dressings was observed using fibroblast cells both before and after hydrogel deposition (FIG. 16). Untreated Acticoat™ presented the highest toxicity to fibroblast cells (47.6±5.1% viable cells) whereas the Acticoat™-PAM1 dressing appeared to be significantly less toxic (60.3±2.6% viable cells, P<0.01).

As demonstrated, hydrogel deposition on commercial dressings, such as Acticoat™ and Silverlon™, can significantly decrease adherency while preserving antibacterial efficacy. As well, cytotoxicity of Acticoat™ Flex 3 to fibroblasts was reduced after hydrogel deposition.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A method for forming a hydrogel coating on a wound dressing, comprising:
(a) providing a substrate, wherein the substrate is a wound dressing;
(b) treating the substrate with O₂ plasma for between about 10 min to about 20 min to produce peroxide functional groups on the substrate surface;
(c) loading a hydrogel precursor solution onto the plasma-treated substrate, the precursor solution comprising acrylamide monomers or acrylic acid monomers, or a combination of acrylamide and acrylic acid monomers;
(d) sandwiching the loaded substrate between two plates, wherein the two plates are positioned to define the thickness of the hydrogel therebetween; and
(e) exposing the loaded substrate to ultraviolet irradiation for about 10 min to about 70 min, wherein the ultraviolet irradiation is at an intensity of about 3 mw/cm2 to about 120 mw/cm2 UVA,
wherein the hydrogel precursor solution is cured onto the substrate to form the hydrogel coating.

2-67. (canceled)

68. The method of claim 1, wherein the wound dressing comprises an antimicrobial additive, a wound-healing additive, or an antimicrobial and a wound-healing additive.

69. The method of claim 68, wherein the antimicrobial additive is silver.

70. The method of claim 68, wherein the wound-healing additive is a growth factor.

71. The method of claim 1, wherein the hydrogel precursor solution comprises acrylamide monomers and N,N′-methylene bisacrylamide as cross-linking agents in a weight ratio from about 90:10 to about 99:1.

72. The method of claim 1, wherein one or more antimicrobial agents, one or more wound-healing agents, or one or more antimicrobial agents and one or more wound-healing agents, are further applied to the hydrogel coating on the coated substrate.

73. The method according to claim 1, wherein one or more antimicrobial agents, one or more wound-healing agents, or one or more antimicrobial agents and one or more wound-healing agents, are loaded onto the plasma-treated substrate with the precursor solution in step (c).

74. A wound dressing comprising a hydrogel coating on a surface of the wound dressing, wherein the hydrogel coating comprises polymerized monomeric derivatives of acrylic acid, said polymerized monomers co-polymerized onto the surface of the wound dressing.

75. The wound dressing of claim 74, wherein the monomers comprise acrylamide monomers, acrylic acid monomers, or a combination of acrylamide and acrylic acid monomers.

76. The wound dressing of claim 75, wherein the monomers comprise acrylamide monomers and N,N′-methylene bisacrylamide as cross-linking agents.

77. The wound dressing of claim 76, wherein the acrylamide monomers and the N,N′-methylene bisacrylamide are in a weight ratio from about 90:10 to about 99:1.

78. The wound dressing of claim 74, wherein the wound dressing additionally comprises an antimicrobial additive, a wound-healing additive, or an antimicrobial and a wound-healing additive.

79. The wound dressing according to claim 78, wherein the wound-healing additive is a growth factor.

80. The wound dressing according to claim 78, wherein the antimicrobial additive is silver.

81. The wound dressing of claim 74, wherein the hydrogel coating is about 20% to about 38% of the total weight of the wound dressing.

82. The wound dressing according to of claim 74, wherein the hydrogel coating has a swelling ratio of about 152% to about 365%.
